SEQUENCE LISTING (4 sequences)

SEQ ID NO: 1 - DNA, 1450 bp, synthetic construct
  source 1..1450: mol_type = other DNA
gccggggggggtgcctatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaagtgaccg

SEQ ID NO: 2 - DNA, 1190 bp, synthetic construct
  source 1..1190: mol_type = other DNA
ctcagggagctggagcatgcatacggtcatgcatgcactgtgcagtaggggcgtctgtcgtaaacgccttgtcgaccactcttgacgttacggttcatcgtgacggaaaaatccactatcaggcgtacgagcgcggtgtacctgtggccgatcttgaagtgatcggtgatactgataagaccggaacgattacgcacttcgttccggatccggaaattttcaaagaaacaaccgaatacgactatgacctgctttcaaaccgtgtccgggaattggccttcctgacaaaaggtgtaaacatcacgattgaagacaaacgtgaaggacaagaacggaaaaacgagtaccactacgaaggcggaatcaaaagctatgttgagtacttaaaccgttccaaagaagtcgttcatgaagagccgatttatatcgaaggcgagaaagacggcataacggttgaagttgcattgcaatacaacgacagctatacaagcaatatttattctttcacaaataatatcaacacatacgaaggcggcacgcacgaagccggatttaaaaccggtctgacccgtgttataaacgactatgcaagaagaaaagggattttcaaagaaaatgatccgaatttaagcggggatgatgtgagggaagggctgactgccattatttcaattaagcaccctgatccgcaattcgaagggcagacgaaaacgaagctcggcaactccgaagcgagaacgatcactgatacgctgttttcttctgcgctggaaacattccttcttgaaaatccggactcagcccgcaaaatcgttgaaaaaggtttaatggccgcaagagcgcggatggcagcgaaaaaagcgcgggaattgacccgccgcaaaagtgcgcttgagatttccaatctgccgggcaaactggcggactgttcttctaaagatccgagcatttccgagctgtatatcgtagagggtgactctgcgggcggatcagcgaaacagggacgggaccgtcatttccaagccattctgccgctgcgcggtaagattctgaacgttgagaaagccagacttgataagattctctcaaacaatgaggtcagatcaatgatcacggccctcggaacaggaatcggagaagattttaatcttgaaaaagcgcgtatccgtaacctgtaatgaagtaccaag

SEQ ID NO: 3 - DNA, 1015 bp, synthetic construct
  source 1..1015: mol_type = other DNA
gggggttacgacggcagtgcagcttgcatgcctgcaggtcgacgattgacgtgggatggctacaactatgaggatgccatcatcatgagtgaacgcctagtgaaggatgatgtttatacatctatccacattgaagaatacgaatcagaagcacgtgatacgaaacttggacctgaagaaatcactcgcgatattccaaacgtcggtgaagatgcgcttcgcaatcttgatgaccgcggaatcatccgtattggggcagaagtaaaagacggagatcttcttgttggtaaagtaacgcctaaaggcgtaactgaactgactgcagaagaacgccttcttcacgccatctttggcgagaaagcccgcgaggttcgtgatacttctcttcgtgtgcctcatggcggcggcggaattatccatgacgttaaagtcttcaaccgtgaagacggagacgaacttcctccaggtgttaaccagttagtacgcgtatatatcgttcagaaacgtaagatttctgaaggggataaaatggccggtcgtcacggtaacaaaggtgttatctctaagattcttcctgaagaagatatgccttaccttcctgacggcacaccaattgatatcatgcttaacccgctgggcgtaccatcacgtatgaacatcgggcaggtattggaacttcacatgggtatggccgctcgttaccttggcattcacattgcatctcctgtatttgacggagcgcgagaagaggatgtctgggaaacacttgaagaagccggcatgtctcgtgacgccaaaacagtgctttacgacggacgtactgagagccgtttgataaccgtgtatctgtcggtatcatgtacatgatcaaactggctcacatggttgacgataaacttcatgcacgctctacaggcccttactcacttgttacgcagcagcctctgcgtaagcgcattgcgacagcgtttgtgagatgagtttggcactgagctacggtgcgcttacactcttc

SEQ ID NO: 4 - DNA, 1256 bp, synthetic construct
  source 1..1256: mol_type = other DNA
cataatgacaactttgtggtaacgggctttctcaaggttgaagtcttccccaatacctgtgccgagcgctgtgatcatagagcgaacttcgttgttagaaaggattttatccagtctggccttttcaacgtttaggattttacctctaagcggcaaaatggcttggaaacgtctgtcgcgtccttgtttagcagatcctccggcagagtcaccctctacgatatataacccggaaatgctcggatcttttgaagagcagtccgctaacttaccgggcaagtttgaaatttccaaagcactcttacggcgtgttagttcacgcgcttttttcgcagccattcttgctcttgccgccattaagcctttatcgacaatttttttggccgcatctggattttccagcataaatgtttccatcgccgtagaaaataacgtatcggtgattgtccgtgcttctgagttgcccagctttgttttcgtttggccctcaaactgcggatcagggtgtttgattgaaataatcgctgtcagcccttcccttacgtcatctccgcttaggtttggatcattttctttaataagccctttttttctggcgtaatcgttgataacacgagtcaggcccgttttgaagccagcttcatgggtaccgccttcgtacgtgttaatgttgtttgtaacgagtaaatgttgcttgtgtagctgtcattgtattgcaaagccacttcaaccgtaatgccgtccttttcgccttcaatgtaaatcggctcttcatggacaacctctttagagcggtttaaatactctacataacttttaattccgccttcgtaatggtattcatttttgcgctcttgtccttcacgtttatcttcaatcgtgatgtttacgccctttgttaaaaaggccaattcacgcacgcggttggcaagcagatcgtaatcatactcggttgtttctgagaatatttcagggtccgggacaaaatgtgtcgtcgttcctgtatgatccgtttcgccaatgatttcaaggtctgtaaccggaactccgcgtttataggtttggcggtgaattttaccgtcacggtgaaccgtcacatcaagctctgttgatagtgcgtttacgaccgacgcacctacaccgtgtaatcctccggatactttatagccgcttccgtcgaacttccccccggcgtgcagaacggtcatgaagacttcaatctctagaggatccccgggtaccgagctcgaattcgt

(19) United States
(12) Patent Application Publication
(10) Pub. No.: US 2025/0327020 A1
(43) Pub. Date: Oct. 23, 2025

(54) BACILLUS VELEZENSIS AND USE THEREOF

(71) Applicants: GUIZHOU UNIVERSITY, Guiyang (CN); CHINA AGRICULTURAL UNIVERSITY, Beijing (CN)

(72) Inventors: Fuyu YANG, Beijing (CN); Danyang ZHAO, Beijing (CN); Kuikui NI, Beijing (CN); Xuekai WANG, Beijing (CN); Yunhong LI, Beijing (CN); Xueting YAN, Beijing (CN)

(73) Assignees: GUIZHOU UNIVERSITY, Guiyang (CN); CHINA AGRICULTURAL UNIVERSITY, Beijing (CN)

(21) Appl. No.: 18/735,444

(22) Filed: Jun. 6, 2024

(30) Foreign Application Priority Data
Apr. 19, 2024 (CN) 202410479522.8

(51) Int. Cl. C12N 1/20; C12R 1/07

(52) U.S. Cl. CPC C12N 1/205; C12R 2001/07

(57) ABSTRACT

The present disclosure relates to Bacillus velezensis (B. velezensis) and a use thereof, and belongs to the technical field of microorganisms. The present disclosure solves one of the problems of the Bacillus in the art such as poor broad-spectrum activity for fungi and bacteria, low inhibitory activity, and limited inhibition range. The B. velezensis QMHF-G5 has an excellent inhibitory activity for pathogenic fungi and bacteria, and can inhibit a variety of pathogenic fungi and bacteria, with a prominent broad-spectrum activity. In addition, the B. velezensis has a high inhibitory activity for a variety of pathogenic fungi and bacteria detected, with a high inhibition rate.

### CROSS-REFERENCE TO RELATED APPLICATION

This application claims priority to Chinese Patent Application No. 202410479522.8, filed on Apr. 19, 2024, the disclosure of which is hereby incorporated by reference in its entirety.

### SEQUENCE LISTING

The instant application contains a Sequence Listing which has been submitted electronically in XML format and is hereby incorporated by reference in its entirety. Said XML copy, created on May 23, 2024, is named BACILLUS VELEZENSIS AND USE THEREOF.xml and is 9,385 bytes in size.

### BACKGROUND

Bacteria of Bacillus, a class of aerobic or facultative anaerobic bacteria, can not only produce endospores in adverse circumstances to resist adverse conditions, but also secrete a variety of beneficial substances to provide a powerful biological control effect. The bacteria of Bacillus are widely distributed in different habitats and are known for producing a wide variety of antagonistic compounds. 5% to 8% of a genome of the bacteria of Bacillus is dedicated to the biosynthesis of secondary metabolites.

In the control of animal and plant diseases, main representative species of the bacteria of Bacillus include Bacillus subtilis (B. subtilis), Bacillus licheniformis, and Bacillus amyloliquefaciens (B. amyloliquefaciens). These bacteria can effectively fight against a variety of pathogens and play an important role in the development of biological pesticides. Studies have shown that B. velezensis also exhibits a broad disease prevention potential and can produce a variety of compounds including iturin, fengycin, and surfactin, and these compounds have significant inhibitory effects on various pathogenic bacteria and fungi, such as Listeria monocytogenes, Aspergillus, and Fusarium. Applications of B. velezensis in corn have shown that B. velezensis can not only inhibit the growth of these pathogenic fungi, but also reduce the production of harmful substances such as aflatoxins and ochratoxins. B. subtilis MBI600, as a type of plant growth-promoting rhizobacteria (PGPR), has been commercialized for promoting the growth of tomato and fighting against soil-borne pathogens. B. subtilis C3 has been used to control the rot of lily bulbs, which demonstrates the application of biological control in the protection of high-value crops. These examples illustrate a dual action of Bacillus in the disease management to directly inhibit pathogens and activate a defense mechanism of a plant.

Bacillus is considered to be an eco-friendly biological control agent, and this biological control agent can regulate a response of a plant to a stress through a variety of hormonal and enzymatic mechanisms, is conducive to soil remediation, and can serve as a denitrification agent in agriculture. Studies have shown that the combination of Bacillus with nanoparticles, such as coating Bacillus with gold, aluminum, or silver nanoparticles, can promote the growth of a plant and prevent the growth of harmful fungi in a rhizosphere zone, which further confirms a potential of Bacillus to serve as a nano-biological control agent. These properties of Bacillus are used not only in the traditional agricultural production, but also in the modern eco-friendly disease management strategies.

However, the currently disclosed Bacillus has problems such as inhibition on only a small number of types of fungi and bacteria, low inhibitory activity, poor broad-spectrum activity, and limited inhibition range.

### SUMMARY

In view of the above analysis, the present disclosure is intended to provide B. velezensis and a use thereof, so as to solve one of the problems of the Bacillus in the art such as poor broad-spectrum activity for fungi and bacteria, low inhibitory activity, and limited inhibition range.

The present disclosure relates to the technical field of microorganisms, and in particular to Bacillus velezensis (B. velezensis) and a use thereof.

In a first aspect, the present disclosure provides B. velezensis QMHF-G5 with an accession number of CGMCC No. 27713.

Further, the B. velezensis QMHF-G5 includes 16S rDNA shown in SEQ ID NO: 1.

Further, the B. velezensis QMHF-G5 includes a QMHF-G5gyrB gene sequence shown in SEQ ID NO: 2.

Further, the B. velezensis QMHF-G5 is isolated from an intestinal tract of Vespula flaviceps.

Further, the B. velezensis QMHF-G5 has an inhibition zone width of more than or equal to 16.74 mm for Aspergillus tubingensis, an inhibition zone width of more than or equal to 14.27 mm for Cochliobolus sativus (Ito & Kurib.) Drechsler, an inhibition zone width of more than or equal to 14.12 mm for Fusarium graminearum, an inhibition zone width of more than or equal to 13.93 mm for Botrytis cinerea, and an inhibition zone width of more than or equal to 12.97 mm for Valsa mali Miyabe et Yamada.

Further, the B. velezensis QMHF-G5 has an inhibition zone diameter of more than or equal to 38.51 mm for Micrococcus flavus and an inhibition zone diameter of more than or equal to 34.62 mm for Paenibacillus larvae.

In a second aspect, the present disclosure provides a biological agent or inhibition agent for inhibiting fungi and bacteria, including the B. velezensis QMHF-G5 described above.

Further, the fungi include Botrytis cinerea, Fusarium graminearum, Colletotrichum orbiculare (Berk. & Mont.) Arx, Phomopsis asparagi, Rhizoctonia cerealis Vander Hoeven, Cochliobolus sativus (Ito & Kurib.) Drechsler, Valsa mali Miyabe et Yamada, Exserohilum turcicum, Gibberella zeae (Schw.) Petch., Macrohoma kawatsukai Hara, Fusarium redolens, Fusarium solani, Fusarium Oxysporum, Magnaporthe grisea Barr, Alternaria panax, Ustilaginoidea virens, Aspergillus tubingensis, Ascosphaera apis, Fusarium pseudocircinatum, and Mucor irregularis. Further, the bacteria include Xanthomonas oryzae pv. oryzae, Pseudomonas solanacearum Smith, Xanthomonas campestris pv malvacearum (E F Smith) Dowson, Paenibacillus larvae, and Micrococcus flavus.

In a third aspect, the present disclosure provides a use of the B. velezensis QMHF-G5 described above in preparation of a drug for controlling animal and plant diseases.

Compared with some implementations, the present disclosure can allow at least one of the following beneficial effects:

(1) The present disclosure discloses B. velezensis QMHF-G5. The B. velezensis QMHF-G5 has an excellent inhibitory activity for pathogenic fungi and bacteria, and can inhibit a variety of pathogenic fungi and bacteria, with a prominent broad-spectrum activity. In addition, the B. velezensis has a high inhibitory activity for a variety of pathogenic fungi and bacteria detected, with a high inhibition rate.

(2) The B. velezensis QMHF-G5 of the present disclosure exhibits an inhibitory effect for 25 plant (or bee) pathogenic fungi and bacteria. In particular, the B. velezensis QMHF-G5 has an inhibition zone width of more than or equal to 16.74 mm for Aspergillus tubingensis, an inhibition zone width of more than or equal to 14.27 mm for Cochliobolus sativus (Ito & Kurib.) Drechsler, an inhibition zone width of more than or equal to 14.12 mm for Fusarium graminearum, an inhibition zone width of more than or equal to 13.93 mm for Botrytis cinerea, and an inhibition zone width of more than or equal to 12.97 mm for Valsa mali Miyabe et Yamada, and the B. velezensis QMHF-G5 has an inhibition zone diameter of more than or equal to 38.51 mm for Micrococcus flavus and an inhibition zone diameter of more than or equal to 34.62 mm for Paenibacillus larvae.

(3) The B. velezensis QMHF-G5 of the present disclosure has a huge application potential in the control and prevention of various diseases caused by fungi and bacteria. With the in-depth research on the action mechanism, safety, and application effect of the B. velezensis QMHF-G5, the B. velezensis QMHF-G5 is expected to become an important microorganism against a variety of pathogens and make important contributions to the human health and environmental protection.

The above technical solutions in the present disclosure can also be combined with each other to provide increased preferred combination solutions. Other features and advantages of the present disclosure will be described in the following description, and some of these will become apparent from the description or be understood by implementing the present disclosure. The objectives and other advantages of the present disclosure may be implemented or derived by those specifically indicated in the description and accompanying drawings.

### BRIEF DESCRIPTION OF THE DRAWINGS

The accompanying drawings are provided merely to illustrate the specific embodiments, rather than to limit the present disclosure. The same reference numerals represent the same components throughout the accompanying drawings.

FIG. 1 shows morphologies of colonies produced after cultivating the strains QMHF-G5 and MS-E23 of the present disclosure;

FIG. 2 shows starch hydrolysis test results of the strains QMHF-G5 and MS-E23 of the present disclosure;

FIG. 3 shows oil hydrolysis test results of the strains QMHF-G5 and MS-E23 of the present disclosure;

FIG. 4 shows gelatin hydrolysis test results of the strains QMHF-G5 and MS-E23 of the present disclosure;

FIG. 5 shows casein hydrolysis test results of the strains QMHF-G5 and MS-E23 of the present disclosure;

FIG. 6 shows D-glucose fermentation test results of the strains QMHF-G5 and MS-E23 of the present disclosure;

FIG. 7 shows D-fructose fermentation test results of the strains QMHF-G5 and MS-E23 of the present disclosure;

FIG. 8 shows sucrose fermentation test results of the strains QMHF-G5 and MS-E23 of the present disclosure;

FIG. 9 shows D-mannitol fermentation test results of the strains QMHF-G5 and MS-E23 of the present disclosure;

FIG. 10 shows hydrogen sulfide test results of the strains QMHF-G5 and MS-E23 of the present disclosure;

FIG. 11 shows indole test results of the strains QMHF-G5 and MS-E23 of the present disclosure;

FIG. 12 shows methyl red test results of the strains QMHF-G5 and MS-E23 of the present disclosure;

FIG. 13 shows V-P test results of the strains QMHF-G5 and MS-E23 of the present disclosure;

FIG. 14 shows catalase test results of the strains QMHF-G5 and MS-E23 of the present disclosure;

FIG. 15 shows Gram staining results of the strains QMHF-G5 and MS-E23 of the present disclosure;

FIG. 16 shows spore staining results of the strains QMHF-G5 and MS-E23 of the present disclosure;

FIG. 17 shows states of the strains QMHF-G5 and MS-E23 of the present disclosure after being stab-inoculated in a LB medium and cultivated for 48 h;

FIG. 18 shows a polymerase chain reaction (PCR) electrophoresis pattern of 16S rDNA of the strains QMHF-G5 and MS-E23 of the present disclosure; and

FIG. 19 shows a PCR electrophoresis pattern of a gyrB gene of the strains QMHF-G5 and MS-E23 of the present disclosure.

### DETAILED DESCRIPTION

Preferred embodiments of the present disclosure will be specifically described below with reference to the accompanying drawings. The accompanying drawings constitute a part of the present disclosure, and are used together with the embodiments of the present disclosure to explain the principles of the present disclosure rather than limit a scope of the present disclosure.

In a first specific embodiment, the present disclosure discloses B. velezensis QMHF-G5 with an accession number of CGMCC No. 27713, which was deposited in the China General Microbiological Culture Collection Center (CGMCC, Institute of Microbiology Chinese Academy of Sciences NO. 1 West Beichen Road, Chaoyang District, Beijing, China) on Jun. 27, 2023.

Compared with some implementations, the B. velezensis QMHF-G5 provided by the present disclosure has an excellent inhibitory activity for pathogenic fungi and bacteria, and can inhibit a variety of pathogenic fungi and bacteria, with a prominent broad-spectrum activity. In addition, the B. velezensis has a high inhibitory activity for a variety of pathogenic fungi and bacteria detected, with a high inhibition rate. With the in-depth research on the action mechanism, safety, and application effect of the B. velezensis QMHF-G5, the B. velezensis QMHF-G5 is expected to become an important microorganism against a variety of pathogens and make important contributions to the human health and environmental protection.

In a specific embodiment, the B. velezensis QMHF-G5 includes 16S rDNA shown in SEQ ID NO: 1.

In the specific embodiment, the B. velezensis QMHF-G5 includes a QMHF-G5gyrB gene sequence shown in SEQ ID NO: 2.

In the specific embodiment, the B. velezensis QMHF-G5 is isolated from an intestinal tract of Vespula flaviceps.

In the specific embodiment, the B. velezensis QMHF-G5 has an inhibition zone width of more than or equal to 16.74 mm for Aspergillus tubingensis, an inhibition zone width of more than or equal to 14.27 mm for Cochliobolus sativus (Ito & Kurib.) Drechsler, an inhibition zone width of more than or equal to 14.12 mm for Fusarium graminearum, an inhibition zone width of more than or equal to 13.93 mm for Botrytis cinerea, and an inhibition zone width of more than or equal to 12.97 mm for Valsa mali Miyabe et Yamada.

In the specific embodiment, the B. velezensis QMHF-G5 has an inhibition zone diameter of more than or equal to 38.51 mm for Micrococcus flavus and an inhibition zone diameter of more than or equal to 34.62 mm for Paenibacillus larvae.

In a second specific embodiment, the present disclosure discloses a biological agent or inhibition agent for inhibiting fungi and bacteria, including the B. velezensis QMHF-G5 described above.

In the specific embodiment, the fungi include Botrytis cinerea, Fusarium graminearum, Colletotrichum orbiculare (Berk. & Mont.) Arx, Phomopsis asparagi, Rhizoctonia cerealis Vander Hoeven, Cochliobolus sativus (Ito & Kurib.) Drechsler, Valsa mali Miyabe et Yamada, Exserohilum turcicum, Gibberella zeae (Schw.) Petch., Macrohoma kawatsukai Hara, Fusarium redolens, Fusarium solani, Fusarium Oxysporum, Magnaporthe grisea Barr, Alternaria panax, Ustilaginoidea virens, Aspergillus tubingensis, Ascosphaera apis, Fusarium pseudocircinatum, and Mucor irregularis.

In the specific embodiment, the bacteria include Xanthomonas oryzae pv. oryzae, Pseudomonas solanacearum Smith, Xanthomonas campestris pv malvacearum (E F Smith) Dowson, Paenibacillus larvae, and Micrococcus flavus.

In a third specific embodiment, the present disclosure discloses a use of the B. velezensis QMHF-G5 described above in preparation of a drug for controlling animal and plant diseases.

The technical solutions of the present disclosure are further described below with reference to specific examples.

In the present disclosure, Vespula flaviceps was collected from Shijingshan District, Beijing on Sep. 15, 2022; and alfalfa was collected from Zhuozhou City, Hebei Province on Aug. 10, 2022.

### Example 1 Isolation and Preliminary Screening of Strains QMHF-G5 and MS-E23

### 1. Isolation and Purification of the Strains

(1) Isolation of the strain QMHF-G5: 2 to 3 Vespula flaviceps individuals were collected and placed in a sterile 1.5 mL centrifuge tube, 600 μL of sterile normal saline was added to the centrifuge tube, the Vespula flaviceps individuals were ground with a grinding rod to obtain a homogenate, the homogenate was vortexed for thorough mixing and then allowed to stand slightly to obtain a turbid solution, and the turbid solution was taken and 10-fold diluted with sterile normal saline serially to 10⁻⁵. 100 μL of each of samples at 10⁻¹ to 10⁻⁵ dilutions was taken and coated on an LB medium plate, and 3 replicates were set for each dilution. Coated plates were cultivated at 37° C. for 24 h to 48 h.

(2) Isolation of the strain MS-E23: 20.0 g of alfalfa leaves was taken and placed in a 250 mL sterile Erlenmeyer flask, 180 mL of sterile normal saline was added to the Erlenmeyer flask, and the Erlenmeyer flask was shaken in a shaker at 4° C. and 240 rpm for 2 h. The alfalfa leaves were soaked in 75% ethanol for 90 s, a 3.25% sodium hypochlorite solution for 120 s, and 75% ethanol for 30 s successively to allow surface disinfection, and then rinsed three times with sterile distilled water to allow surface sterilization. The alfalfa leaves with surfaces sterilized were transferred to a sterile mortar and ground to obtain a ground material, and 45 mL of normal saline was added to the mortar and thoroughly mixed with the ground material to obtain a suspension of microorganisms in the leaves. The suspension was 10-fold diluted with sterile normal saline serially to 10⁻⁵. 100 μL of each of samples at 10⁻¹ to 10⁻⁵ dilutions was taken and coated on an LB medium plate, and 3 replicates were set for each dilution. Coated plates were cultivated at 37° C. for 24 h to 48 h.

According to parameters such as sizes, shapes, edges, glosses, textures, colors, and transparencies of colonies, colonies from the above two different strains were streaked on LB medium plates for purification to obtain pure bacterial cultures, and the different strains were numbered and then stored in 20% glycerol at −80° C.

TABLE 1
Sample collection information

| | Collection | | |
|---|---|---|---|
| Sample source | time | Collection site | Strain No. |
| Intestinal tract of | 2022 Sep. | Shijingshan District, | QMHF- |
| Vespula flaviceps | 15 | Beijing | G5 |
| Endophytes of | 2022 Aug. | Zhuozhou City, | MS-E23 |
| alfalfa | 10 | Hebei Province | |

### 2. Preliminary Screening of the Strains QMHF-G5 and MS-E23

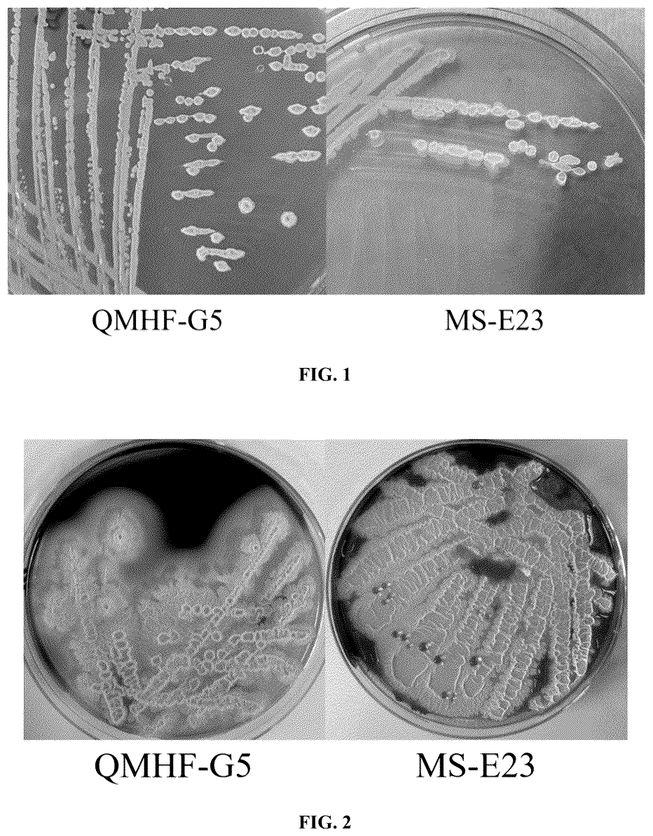

Milky-white or light-yellow colonies with round or irregular shapes and smooth or wavy edges were picked from the LB medium plates, streaked on an LB solid medium for purification, and cultivated at 37° C. for 48 h. Morphologies of resulting colonies are shown in FIG. 1, and it can be seen that the colonies are light-yellow and have tight textures, irregular edges, ring-shaped bulges in the middle, and folds in rings, which are denoted as the strain QMHF-G5.

The strain MS-E23 was cultivated on an LB solid medium at 37° C. for 48 h. Morphologies of resulting colonies are shown in FIG. 1, and it can be seen that the colonies are milky-white and have irregular edges, moist textures, and ring-shaped bulges in the middle.

### 3. Identification of the Strains

### (I) Physiological and Biochemical Identification of the Strains

### (1) Starch Hydrolysis Test

A fresh solid culture of a strain to be identified was streaked on a starch medium plate, the starch medium plate was invertedly incubated at 37° C. for 48 h, and after obvious colonies were formed, an iodine solution was added dropwise to the plate, and the plate was blue-black. If there is a transparent zone around a colony, it indicates a positive starch hydrolysis result; and if there is still blue-black around a colony, it indicates a negative starch hydrolysis result.

A starch medium (an LB medium+1.0% of a soluble starch) (100 mL) was prepared from the following raw materials: 1.0 g of tryptone, 0.5 g of a yeast extract, 1.0 g of NaCl, 1.0 g of the soluble starch, 1.5 g of agar, and 100 mL of distilled water. The starch medium was incubated in a water bath for melting, autoclaved at 121° C. for 20 min, and then poured into a plate for later use.

Results: Starch hydrolysis test results of the strains QMHF-G5 and MS-E23 are shown in FIG. 2. After the strain QMHF-G5 is cultivated on the starch medium for 48 h and then the iodine solution is added dropwise around colonies, a transparent zone is formed around the colonies, indicating that the strain can secrete an amylase to hydrolyze the starch. Therefore, the strain QMHF-G5 has a positive starch hydrolysis result. A transparent zone is also formed around the strain MS-E23, indicating that the strain can also secrete an amylase to hydrolyze the starch. Therefore, the strain MS-E23 also has a positive starch hydrolysis result.

### (2) Oil Hydrolysis Test

A melted solid oil medium was cooled to about 50° C., thoroughly shaken to make oil evenly distributed, and poured into a plate. A strain to be identified was streaked on an oil medium plate, the oil medium plate was invertedly incubated at 37° C. for 24 h, and a color of the bacterial lawn was observed. If a red spot appears, it indicates a positive oil hydrolysis result.

An oil medium (100 mL) was prepared from the following raw materials: 1.0 g of a peptone, 0.5 g of a beef extract, 0.5 g of NaCl, 1.0 g of peanut oil or sesame oil, 0.1 mL of a 1.6% neutral red aqueous solution, 1.5 g of agar, and 100 mL of distilled water. The oil medium had a pH of 7.2, and was autoclaved at 121° C. for 20 min and then poured into a plate for later use.

Notes: Spoiled oil could not be used, the oil, agar, and distilled water were heated first, and the neutral red was added after the pH was adjusted. The 1.6% neutral red aqueous solution was prepared from the following raw materials: 1.6 g of neutral red, 28 mL of 95% ethanol, and 72 mL of distilled water.

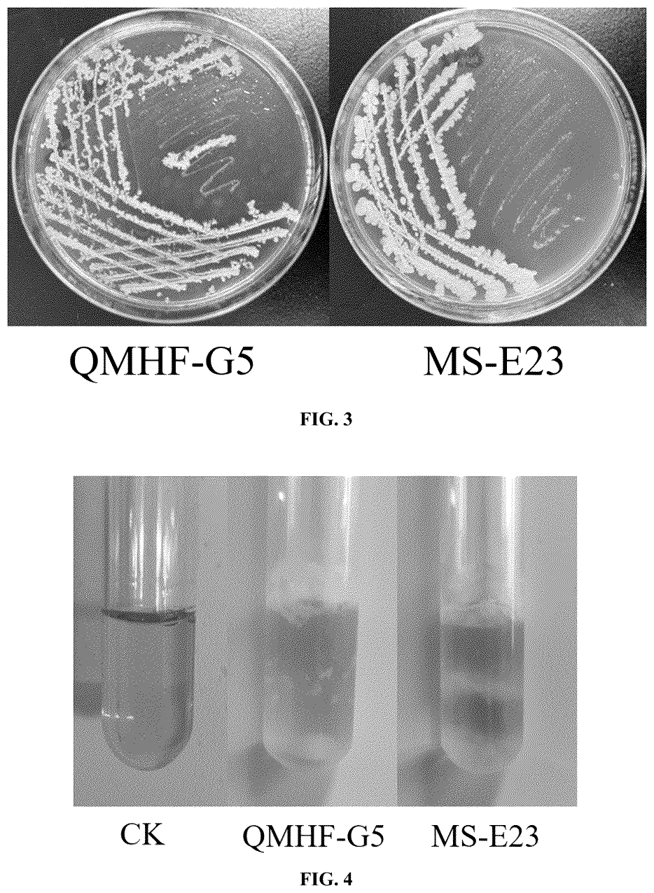

Results: Oil hydrolysis test results of the strains QMHF-G5 and MS-E23 are shown in FIG. 3. No red spots appear around colonies of the strain QMHF-G5, indicating that the strain cannot produce lipase and has a negative lipase hydrolysis result. No red spots appear around colonies of the strain MS-E23, indicating that the strain cannot produce lipase and has a negative lipase hydrolysis result.

### (3) Gelatin Hydrolysis Test:

A gelatin medium test tube was taken, a strain to be identified was stab-inoculated into the gelatin medium test tube by an inoculation needle, and an inoculated test tube was incubated at 20° C. for 2 d to 5 d. The liquefaction of the gelatin was observed.

A gelatin medium (100 mL) was prepared from the following raw materials: an LB medium, and 12 g to 18 g of a gelatin. The gelatin medium had a pH of 7.2 to 7.4. The above raw materials were mixed and melted in a water bath to obtain a first mixed system, then a pH of the first mixed system was adjusted to 7.2 to 7.4 to obtain a second mixed system, and the second mixed system was dispensed into test tubes and autoclaved at 121° C. for 20 min.

Results: Gelatin hydrolysis test results of the strains QMHF-G5 and MS-E23 are shown in FIG. 4. After the strains QMHF-G5 and MS-E23 are stab-inoculated into the gelatin medium and cultivated at 20° C. for 3 d to 4 d, the liquefaction of the gelatin medium is observed. Therefore, the strains QMHF-G5 and MS-E23 both can produce a gelatinase to hydrolyze the gelatin, indicating a positive gelatin hydrolysis result.

### (4) Casein Hydrolysis Test (Litmus Milk Test)

A strain to be identified was inoculated into a litmus milk medium test tube and cultivated at 37° C. for 24 h to 48 h. If a litmus milk medium becomes clear, it indicates a positive result.

A litmus milk medium was prepared as follows: 100 mL of a skimmed milk and 4 mL of a 2.5% litmus aqueous solution (which was placed overnight and then filtered before use) were mixed to obtain a lilac or moderately-purple mixed solution, and the mixed solution was dispensed into test tubes with a height of 4 cm in each test tube and then autoclaved at 121° C. for 15 min.

Results: Casein hydrolysis test results of the strains QMHF-G5 and MS-E23 are shown in FIG. 5. A litmus milk medium into which no strain is inoculated is adopted as a blank control. After cultivation at 37° C. for 48 h, the litmus milk media into which the strains QMHF-G5 and MS-E23 are inoculated become clear, and the litmus milk medium in the blank control does not change significantly (the litmus milk medium does not become clear), indicating that the strains QMHF-G5 and MS-E23 both have positive casein hydrolysis results and can hydrolyze the casein.

### (5) Sugar and Alcohol Fermentation Test

A strain to be identified that was cultivated for 18 h was inoculated into a sugar fermentation medium and cultivated at 37° C. for 24 h to 48 h to check the results, and a negative control was set. Whether a color in each test tube changed and whether bubbles were produced in each Durham tube were observed. If an indicator changes from purple to yellow, it indicates the fermentation of sugars to produce acids and a positive fermentation result.

A sugar fermentation medium (100 mL) was prepared as follows: 1 g of tryptone, 0.5 g of NaCl, and 0.05 mL of a 1.6% bromocresol purple ethanol solution were mixed to obtain a first mixed solution, a pH of the first mixed solution was adjusted to 7.6 to obtain a second mixed solution, the second mixed solution was diluted to 100 mL and dispensed into test tubes (10 mL/tube), a Durham tube was placed in each test tube with the culture filled in the Durham tube, and then the test tubes were autoclaved at 121° C. for 20 min.

A 20% sugar solution (D-glucose, D-fructose, sucrose, and D-mannitol) was autoclaved at 112° C. for 30 min, and 0.5 mL of the autoclaved 20% sugar solution was aseptically added to each test tube.

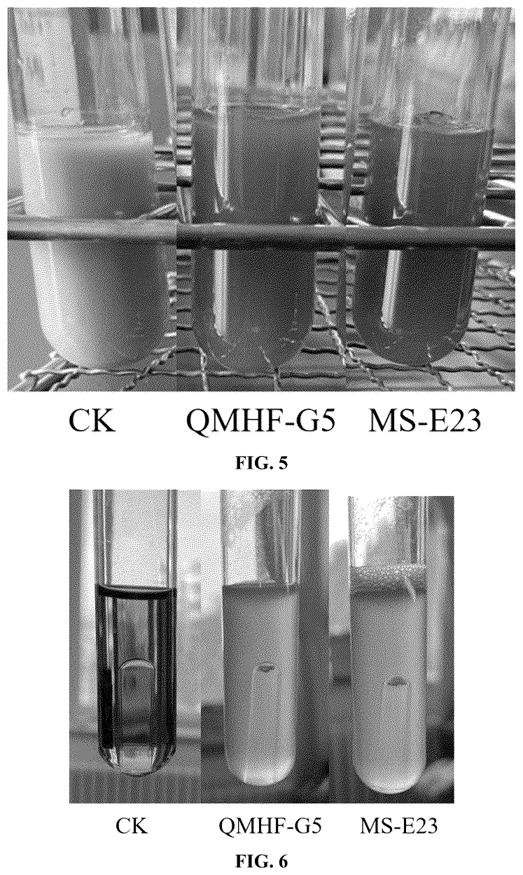

Results: D-glucose fermentation test results of the strains QMHF-G5 and MS-E23 are shown in FIG. 6. Colors of glucose fermentation media into which the strains QMHF-G5 and MS-E23 are inoculated respectively do not change, indicating that D-glucose cannot be decomposed. Bubbles are produced in Durham tubes for the strains QMHF-G5 and MS-E23, indicating that, when the strains QMHF-G5 and MS-E23 ferment D-glucose, a gas is produced and no acid is produced.

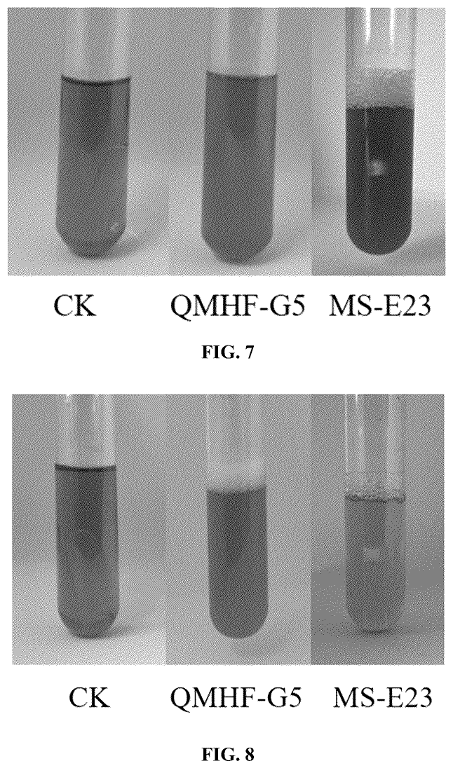

D-fructose fermentation test results of the strains QMHF-G5 and MS-E23 are shown in FIG. 7. A color of a D-fructose fermentation medium into which the strain QMHF-G5 is inoculated does not change, indicating that D-fructose cannot be decomposed. No bubbles are produced in a Durham tube for the strain QMHF-G5, indicating that, when the strain QMHF-G5 ferments D-fructose, neither a gas nor an acid is produced. A color of a D-fructose fermentation medium into which the strain MS-E23 is inoculated does not change, indicating that D-fructose cannot be decomposed. Bubbles are produced in a Durham tube for the strain MS-E23, indicating that, when the strain QMHF-G5 ferments D-fructose, no acid is produced and a gas is produced.

Sucrose fermentation test results of the strains QMHF-G5 and MS-E23 are shown in FIG. 8. A color of a sucrose fermentation medium into which the strain QMHF-G5 is inoculated is purple and does not change, indicating that fructose cannot be decomposed. No bubbles are produced in a Durham tube for the strain QMHF-G5, indicating that, when the strain QMHF-G5 ferments sucrose, neither an acid nor a gas is produced. A color of a sucrose fermentation medium into which the strain MS-E23 is inoculated changes to yellow, indicating that fructose can be decomposed. Bubbles are produced in a Durham tube for the strain MS-E23, indicating that, when the strain MS-E23 ferments sucrose, both an acid and a gas are produced.

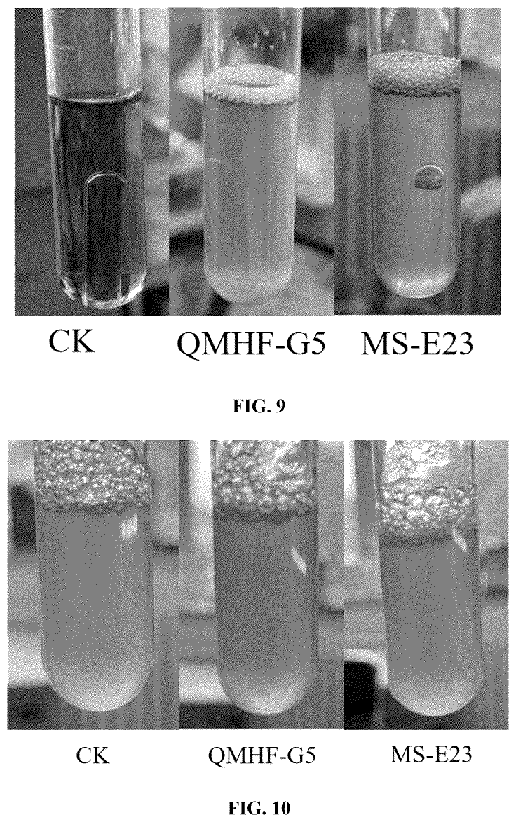

D-mannitol fermentation test results of the strains QMHF-G5 and MS-E23 are shown in FIG. 9. A color of a D-mannitol fermentation medium into which the strain QMHF-G5 is inoculated does not change and is still purple, and no bubbles are produced in a Durham tube for the strain QMHF-G5, indicating that, when the strain QMHF-G5 ferments D-mannitol, neither an acid nor a gas is produced. A color of a D-mannitol fermentation medium into which the strain MS-E23 is inoculated does not change and is still purple, and bubbles are produced in a Durham tube for the strain MS-E23, indicating that, when the strain MS-E23 ferments D-mannitol, no acid is produced and a gas is produced.

### (6) Hydrogen Sulfide Test

A strain to be identified was stab-inoculated into a lead acetate medium with an inoculation needle and cultivated at 37° C. for 48 h. When the lead acetate medium turns black, it indicates a positive result. If there is a negative result, the strain should be further cultivated until the 6th day.

A lead acetate medium (100 mL) was prepared as follows: 0.3 g of a beef extract, 1.0 g of a peptone, 0.5 g of NaCl, and 1.5 g of agar were mixed, diluted to 100 mL, heated for dissolution, and cooled to 60° C. to obtain a first system, then 0.25 g of sodium thiosulfate was added to the first system to obtain a second system, a pH of the second system was adjusted to 7.2 to obtain a third system, the third system was dispensed into Erlenmeyer flasks, sterilized at 115° C. for 15 min, then taken out, and cooled to 55° C. to 60° C. to obtain a fourth system, 1 mL of a 10% lead acetate aqueous solution (sterile) was added to the fourth system to obtain a fifth system, and the fifth system was thoroughly mixed and poured into sterile test tubes.

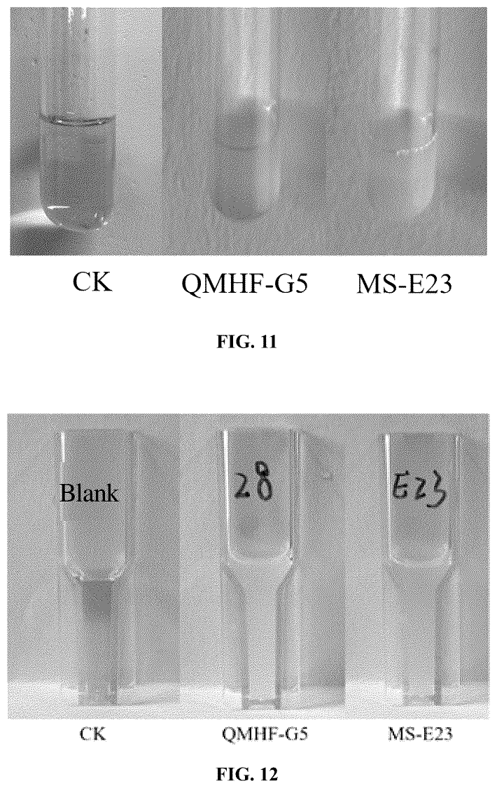

Results: Hydrogen sulfide test results of the strains QMHF-G5 and MS-E23 are shown in FIG. 10. After the strains QMHF-G5 and MS-E23 are stab-inoculated into the lead acetate medium and cultivated at 37° C. for 48 h, the lead acetate medium does not turn black, and after the strains are further cultivated until the 6th day, it still does not turn black near a stab line, indicating that the strains QMHF-G5 and MS-E23 both do not produce hydrogen sulfide and have a negative hydrogen sulfide test result.

### (7) Indole Test

A strain to be identified was inoculated into an indole medium and cultivated at 37° C. for 48 h. 2 drops of an indole reagent were slowly added to a surface of a culture along a tube wall. If a liquid interface turns red, it indicates a positive reaction. When a reaction was not obvious, 4 to 5 drops of diethyl ether were first added to the culture, a resulting system was allowed to stand for 1 min, and after the diethyl ether ascended, the indole reagent was then added and then a color reaction was observed. If a red ring is produced between the diethyl ether and the culture, it indicates a positive reaction.

The indole medium (100 mL) was prepared from the following raw materials: 1.0 g of trypsone, 0.5 g of NaCl, and 0.05 g of L-tryptophan. The indole medium had a pH of 7.6 and was autoclaved at 121° C. for 15 min for later use.

The indole reagent was prepared from the following raw materials: 2 g of p-dimethylaminobenzaldehyde, 190 mL of 95% ethanol, and 40 mL of concentrated HCl.

Results: After the strains QMHF-G5 and MS-E23 each are inoculated into the indole medium and cultivated at 37° C. for 48 h and then 2 drops of the indole reagent are slowly added along a tube wall, liquid interfaces for both of the strains do not turn red, as shown in FIG. 11, indicating that the strains QMHF-G5 and MS-E23 both have a negative indole reaction.

### (8) Methyl Red Test

A strain to be identified was picked and inoculated into a glucose peptone water medium and cultivated at 37° C. Starting from the next day, 1 mL of a culture was collected every day, and 1 to 2 drops of a methyl red indicator were added. A bright-red color indicates a positive result, a light-red color indicates a weakly-positive result, and a yellow color indicates a negative result. When it was positive or it was still negative on the 5th day, a result could be determined.

The glucose peptone water medium (100 mL) was prepared as follows: 0.5 g of a peptone, 0.5 g of glucose, and 0.2 g of K₂HPO₄ were dissolved in 100 mL of water to obtain a first solution, a pH of the first solution was adjusted to 7.0 to 7.2 to obtain a second solution, and the second solution was dispensed into test tubes with 10 mL in each test tube and sterilized at 112° C. for 30 min.

The methyl red indicator was prepared from the following raw materials: 0.04 g of methyl red, 60 mL of 95% ethanol, and 40 mL of distilled water. The methyl red was first dissolved in the ethanol and then the distilled water was added.

Results: After the strains QMHF-G5 and MS-E23 each are inoculated into the glucose peptone water medium and cultivated at 37° C., and take samples after cultivation until the 5th day and the methyl red indicator is added to the samples, bacterial solutions have the same color as the blank control (an uninoculated medium) and are yellow, as shown in FIG. 12. The above results show that the strains QMHF-G5 and MS-E23 both have a negative methyl red reaction result.

(9) V-P test

A strain to be identified was inoculated into a glucose peptone water medium (a V-P assay medium) in a test tube and cultivated at 37° C. for 48 h, then 1 mL of 40% NaOH and 1 mL of 5% α-naphthol were added to the test tube, and the test tube was vigorously shaken and then incubated at 37° C. for 15 min to 30 min to speed up a reaction. If a culture is red, it indicates a positive V-P reaction.

The V-P assay medium (100 mL) was prepared as follows: 0.5 g of a peptone, 0.5 g of glucose, and 0.2 g of NaCl were dissolved in 100 mL of water to obtain a first solution, a pH of the first solution was adjusted to 7.0 to 7.2 to obtain a second solution, and the second solution was dispensed into test tubes with 4 mL to 5 mL in each test tube and sterilized at 112° C. for 30 min.

The 5% α-naphthol was prepared from the following raw materials: 5 g of α-naphthol and 100 mL of absolute ethanol.

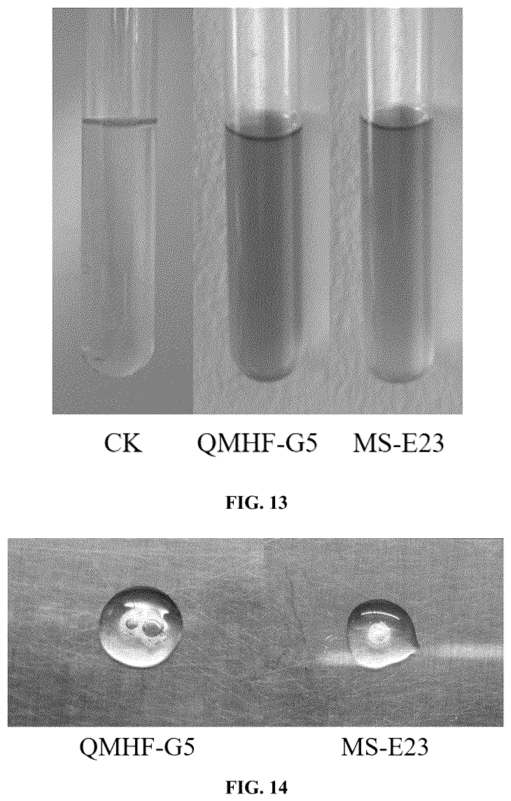

Results: After the strains QMHF-G5 and MS-E23 each are inoculated in the glucose peptone water medium and cultivated at 37° C. for 48 h and then 1 mL of the 40% NaOH and 1 mL of the 5% α-naphthol are added, fermentation broths of the QMHF-G5 and MS-E23 strains both turn red, as shown in FIG. 13, indicating that the strains QMHF-G5 and MS-E23 both have a positive V-P reaction.

### (10) Catalase Test

A small amount of a Bacillus culture was picked from an LB slope and coated on a clean glass slide, and 10% hydrogen peroxide was added dropwise. If bubbles are produced, it indicates a positive result.

Results: After a small amount of a culture is picked from each of plates on which the strains QMHF-G5 and MS-E23 are streaked respectively for purification and coated on a clean glass slide and then 10% hydrogen peroxide is added dropwise, bubbles are produced, as shown in FIG. 14. Therefore, the strains QMHF-G5 and MS-E23 both have a positive catalase reaction.

### (II) Gram Staining (Gram Staining Kit)

Gram staining was conducted with a Solarbio Gram Staining Kit (Cat. No. G1060). Specific operation steps were as follows:

(1) The strains QMHF-G5 and MS-E23 cultivated on plates each were picked, inoculated into an LB test tube, and cultivated at 37° C. under shaking for 24 h.

(2) Smear fixation: 10 μL of a bacterial solution was taken and added to a glass slide, and the glass slide was allowed to pass through a flame 1 to 2 times for fixation to obtain a smear, where the overheating should be avoided.

(3) Staining: The crystal violet was added to the smear to allow staining for 1 min, and then the smear was washed with water. An iodine solution was added to the smear to allow staining for 1 min, and then the smear was washed with water. A destaining solution was added to the smear, the smear was shaken, the destaining was conducted for about 20 s to 60 s according to a thickness of the smear, then the smear was washed with water, and the water was suck-dried. The safranin was added to the smear to allow staining for 1 min, then the smear was washed with water, and the smear was suck-dried or air-dried and then observed under oil immersion lens. Gram-negative bacteria would be stained red, and Gram-positive bacteria would be stained purple.

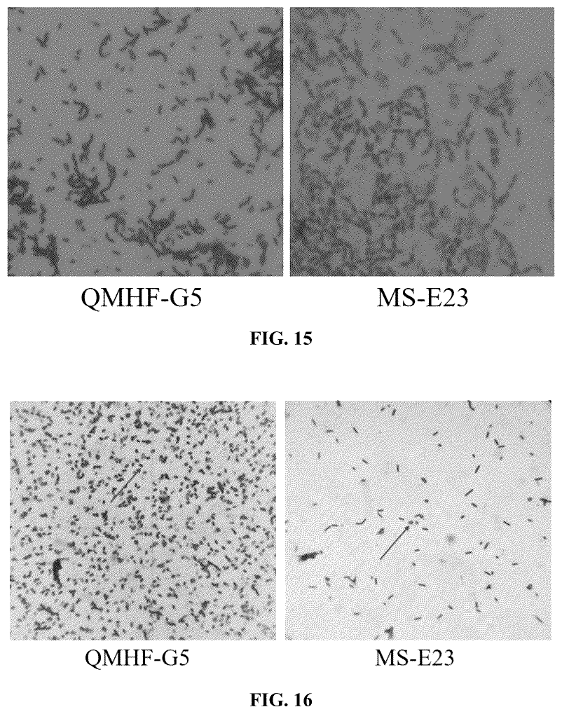

Results: Gram staining results are shown in FIG. 15. Bacterial cells of the strain QMHF-G5 are short rod-shaped and the cells of the strain are purple after Gram staining, indicating that the strain QMHF-G5 is a Gram-positive strain. Bacterial cells of the strain MS-E23 are rod-shaped and the cells of the strain are purple after Gram staining, indicating that the strain MS-E23 is a Gram-positive strain.

### (III) Spore Staining (Spore Staining Kit)

The spore staining was conducted with a spore staining kit (Cat. No. R23109) of Shanghai Yuanye Bio-Technology Co., Ltd. by a Scharffer-Fulton method, namely, a malachite green staining method. Specific operation steps were as follows:

(1) The strains QMHF-G5 and MS-E23 cultivated on plates each were picked, inoculated into an LB test tube, and cultivated at 37° C. under shaking for 48 h.

(2) 10 μL of a bacterial solution was taken and added to a clean and oil-free glass slide to prepare a smear, and the smear was dried naturally.

(3) A malachite green staining solution was added dropwise to the coated solution, and bacteria were allowed to be evenly distributed.

(4) The smear was intermittently heated with a mild fire to allow staining for 10 min.

(5) The smear was gently rinsed with water.

(6) The smear was stained with a spore counterstaining solution for 1 min and then rinsed with water.

Microscopic examination: The smear was dried and then observed under oil immersion lens. Spores were stained green, and bacterial cells were stained red.

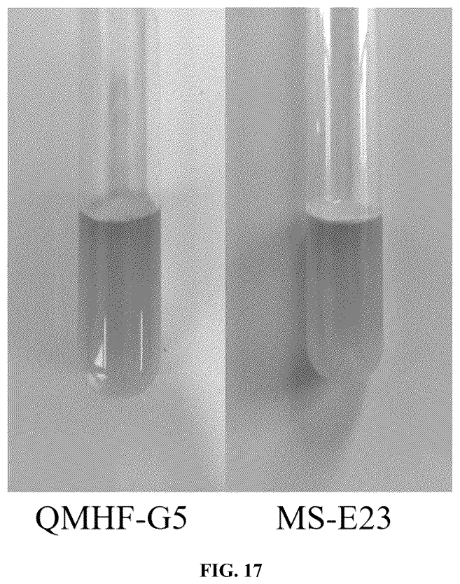

Results: Spore staining results are shown in FIG. 16. In QMHF-G5 cells, spores are stained green and are short and blunt-round, indicating that spores can be produced. In MS-E23 cells, spores are stained green and are short and blunt-round, indicating that spores can also be produced.

### (IV) Motility

The strains QMHF-G5 and MS-E23 each were stab-inoculated into an LB medium by an inoculation needle and cultivated at 37° C. for 48 h. The growth was visually observed every 24 h after the inoculation. If a test strain spreads in a cloud-like manner from a stab line, it indicates that the test strain is motile and positive.

The LB medium (100 mL) was prepared from the following raw materials: 1.0 g of tryptone, 0.5 g of a yeast extract, 1.0 g of NaCl, 1.5 g of agar, and 100 mL of distilled water. The LB medium had a pH of 7.2, and was dispensed into test tubes and autoclaved at 121° C. for 20 min.

Results: After the two Bacillus strains each are stab-inoculated into the LB medium and cultivated for 48 h, the two strains both grow in all directions from a stab line, and both are motile. The strain QMHF-G5 grows in a significant cloud-like manner and has a stronger motility than the strain MS-E23.

In summary, the basic biological characteristics of the strains QMHF-G5 and MS-E23 are shown in Table 2 below.

TABLE 2
Basic biological characteristics
of the strains QMHF-G5 and MS-E23

| | | |
|---|---|---|
| Biological characteristics | QMHF-G5 | MS-E23 |

| Gram staining | | | Positive | Positive |
|---|---|---|---|---|
| Spore | | | Present | Present |
| Motility | | | + | + |
| Starch hydrolysis | | | + | + |
| Oil hydrolysis | | | − | − |
| Gelatin hydrolysis | | | + | + |
| Casein hydrolysis | | | + | + |
| | Acid | D-glucose | − | − |
| | production | D-fructose | − | − |
| | | Sucrose | − | + |
| | | D-mannitol | − | − |

| | | |
|---|---|---|
| H₂S production | − | − |
| Indole production | − | − |
| Methyl red reaction | − | − |
| V-P test | + | + |
| Catalase | + | + |

### (V) Molecular Identification of the Strains

### 1. Extraction of Genomic DNA

Single colonies were picked from a streaked LB plate for activation, inoculated into a fresh LB liquid medium, and cultivated under shaking at 37° C. and 200 rpm until OD₆₀₀ of a resulting bacterial solution was 0.8 to 1.0, and 5 mL of the bacterial solution was taken and centrifuged to collect bacterial cells. Genomic DNA was extracted with a bacterial genomic DNA extraction kit (DC112-01, Vazyme, China).

2. PCR Amplification of 16S rDNA

The genomic DNA was taken as a template and the following primers were designed and synthesized (Sangon Biotech (Shanghai) Co., Ltd., China) for amplification of 16S rDNA:

27F:
5′-AGAGTTTGATCCTGGCTCAG-3′;
and
1492R:
5′-GGTTACCTTGTTACGACTT-3′.

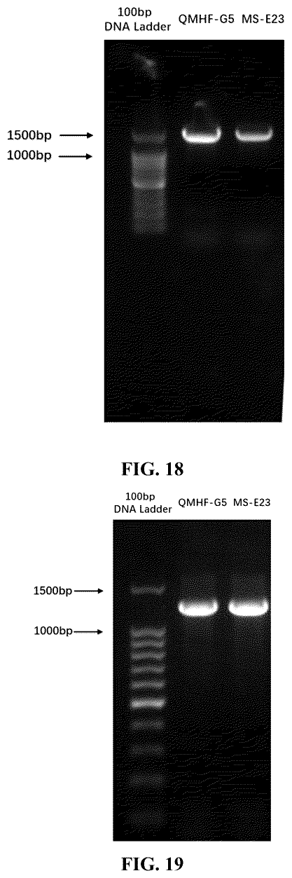

Then, a 16S rDNA fragment was amplified with a Q5 high-fidelity polymerase (BDTP1180, NEB, USA), and an amplified fragment was detected as a single band by 1% agarose gel electrophoresis (the fragment has a length of about 1,500 bp, and the result is shown in a respective figure), and sent to a sequencing company for sequencing (Sangon Biotech (Shanghai) Co., Ltd., China). After 16S rDNA fragment sequences were obtained according to sequencing results, the 16S rDNA fragment sequences each were subjected to alignment analysis by Basic Local Alignment Search Tool (BLAST) (http://www.ncbi.nlm.nih.gov) in GenBank to obtain names of the identified strains similar to the two strains QMHF-G5 and MS-E23, such that the strains were identified. A PCR electrophoresis pattern of 16S rDNA for the 2 strains is shown in FIG. 18.

### A PCR System:

| | | |
|---|---|---|
| Genomic DNA | 50-100 | ng |
| 27F | 0.5 | mM |
| 1492R | 0.5 | mM |
| 2 × Q5 mix | 25 | μL |

| | |
|---|---|
| H₂O | making up to 50 μL |

### Dna Amplification Procedure:

| | | | |
|---|---|---|---|
| 98° C. | 30 s | | |
| 98° C. | 10 s | | |
| 50° C. | 15 s | {close oversize bracket} | 30 cycles |
| 72° C. | 45 s | | |
| 72° C. | 10 min | | |

3. PCR Amplification of a gyrB Gene

The genomic DNA was taken as a template, and the following degenerate primers were designed and synthesized (Sangon Biotech (Shanghai) Co., Ltd., China) for amplification of the gyrB gene (DNA gyrase subunit B gene, partial sequence, with a length of about 1,200 bp), where underlined parts were sequencing primers UP-1S and UP-2Sr, respectively:

gyrB-F:
5′-GAAGTCATCATGACCGTTCTGCAYGCNGGNGGNAARTTYGA-3′;
and
gyrB-R:
5′-AGCACGGTACGGATGTGCGAGCCRTCNACRTCNGCRTCNGCRTCNGT
CAT-3′.

Then, a gyrB gene fragment was amplified with a Q5 high-fidelity polymerase (BDTP1180, NEB, USA), and an amplified fragment was detected as a single band by 1% agarose gel electrophoresis (the fragment has a length of about 1,200 bp, and the result is shown in a respective figure), and sent to a sequencing company for sequencing with the sequencing primers UP-1S and UP-2Sr (Sangon Biotech (Shanghai) Co., Ltd., China). A PCR electrophoresis pattern of the gyrB gene for the 2 strains is shown in FIG. 19.

### A PCR System:

| | | |
|---|---|---|
| Genomic DNA | 50-100 | ng |
| gyrB-F | 0.5 | mM |
| gyrB-R | 0.5 | mM |
| 2 × Q5 mix | 25 | μL |

| | |
|---|---|
| H₂O | making up to 50 μL |

### DNA Amplification Procedure:

| | | | |
|---|---|---|---|
| 98° C. | 30 s | | |
| 98° C. | 10 s | | |
| 55° C. | 15 s | {close oversize bracket} | 30 cycles |
| 72° C. | 36 s | | |
| 72° C. | 10 min | | |

4. Sequence Analysis for the 16S rDNA and gyrB Gene

Sequences obtained after sequencing of the 16S rDNA (as shown in SEQ ID NO: 1) and the gyrB gene (as shown in SEQ ID NO: 2) of the strain QMHF-G5 and the 16S rDNA (as shown in SEQ ID NO: 3) and the gyrB gene (as shown in SEQ ID NO: 4) of the strain MS-E23 were subjected to BLAST alignment in the National Center for Biotechnology Information (NCBI) database. Alignment results are shown in Table 3.

TABLE 3
Molecular identification of the strains QMHF-G5 and MS-E23

| Strain | 16S rDNA (similarity) | gyrB (similarity) |
|---|---|---|
| QMHF- | B. velezensis strain TB6 | B. velezensis strain |
| G5 | (99.93%) | WLYS23 (99.91%) |
| | Bacillus methylotrophicus (B. | B. amyloliquefaciens strain |
| | methylotrophicus) strain B4 | MG-2 (99.91%) |
| | (99.93%) | |
| | B. subtilis strain SYS-Y105 | |
| | (99.86%) | |
| | B. amyloliquefaciens strain BA40 | |
| | (99.72%) | |
| MS-E23 | B. subtilis subsp. subtilis | B. subtilis subsp. subtilis |
| | strain NCD-2 (98.07%) | strain NCD-2 (98.93%) |

It can be seen from Table 3 that a 16S rDNA sequence of the strain QMHF-G5 has a similarity of 99.93% with 16S rDNA sequences of B. velezensis, B. methylotrophicus, and B. subtilis, a similarity of 99.86% with a 16S rDNA sequence of B. subtilis, and a similarity of 99.72% with a 16S rDNA sequence of B. amyloliquefaciens, and a gyrB gene sequence of the strain QMHF-G5 has a similarity of 99.91% with gyrB gene sequences of B. velezensis and B. amyloliquefaciens. In combination with the above physiological and biochemical experimental results, the strain QMHF-G5 was identified as B. velezensis.

A gyrB gene sequence of the strain MS-E23 has a similarity of 98.93% with a gyrB gene sequence of B. subtilis subsp. subtilis. The strain MS-E23 was identified as B. subtilis subsp. subtilis.

### 4. Determination of Inhibitory Activities of the Strains QMHF-G5 and MS-E23

The inhibitory activities of bacilli against pathogenic fungi and bacteria were detected in vitro by a double cultivation method. 18 plant pathogenic fungi, 3 plant pathogenic bacteria, 2 bee pathogenic fungi, 1 bee pathogenic bacterium, and 1 common indicator bacterium (the 25 pathogenic fungi and bacteria were shown in Table 4, the pathogenic fungi and bacteria were provided by the Laboratory of Plant Diseases and Pests, the Institute of Plant Protection, Chinese Academy of Agricultural Sciences and the Laboratory of Bee Diseases and Pests, the Institute of Apicultural Research, Chinese Academy of Agricultural Sciences, and the above strains were isolated from the nature and identified according to the conventional methods known in the art) were taken as target strains. The inhibitory activities of the strains QMHF-G5 and MS-E23 were determined.

TABLE 4
25 plant (or bee) pathogenic fungi and bacteria

| Strain name | Taxonomic status | Disease caused accordingly |
|---|---|---|
| Botrytis cinerea | Botryotinia of | Soybean Botrytis blight |
| | Ascomycota | |
| Fusarium graminearum | Fusarium of | Corn mildew and rot diseases |
| | Deuteromycota | |
| Colletotrichum orbiculare | Colletotrichum of | Cucumber anthracnose |
| (Berk.&Mont.) Arx | Deuteromycotina | |
| Phomopsis asparagi | Sphaeropsidales | Asparagus stem rot |
| Rhizoctonia cerealis Vander | Deuteromycotina | Wheat sharp eyespot |
| Hoeven | | |
| Cochliobolus sativus (Ito & | Cochliobolus of | Wheat root rot |
| Kurib.) Drechsler | Ascomycotina | |
| Valsa mali Miyabe et Yamada | Ascomycotina | Apple valsa canker |
| Exserohilum turcicum | Deuteromycotina | Northern corn leaf blight |
| Gibberella zeae (Schw.) Petch. | Ascomycotina | Wheat Fusarium head blight |
| Macrohoma kawatsukai Hara | Deuteromycotina | Apple ring rot |
| Fusarium redolens | Fusarium of | Alfalfa root rot |
| | Deuteromycotina | |
| Fusarium solani | Fusarium of | Alfalfa root rot |
| | Deuteromycotina | |
| Fusarium Oxysporum | Fusarium of | Soybean root rot |
| | Deuteromycotina | |
| Magnaporthe grisea Barr | Ascomycotina | Rice blast |
| Alternaria panax | Alternaria of | Ginseng black spot |
| | Deuteromycotina | |
| Ustilaginoidea virens | Deuteromycotina | Rice false smut |
| Aspergillus tubingensis | Aspergillus of | Bee aspergillosis |
| | Ascomycota | |
| Ascosphaera apis | Ascosphaera of | Bee chalkbrood |
| | Ascomycota | |
| Fusarium pseudocircinatum | Fusarium of | Sunflower malformation |
| | Ascomycota | |
| Mucor irregularis | Mucor of Zygomycota | Strawberry root rot |
| Xanthomonas oryzae pv. oryzae | Xanthomonas | Rice bacterial leaf blight |
| Pseudomonas solanacearum | Pseudomonas | Peanut bacterial wilt |
| Smith | | |
| Xanthomonas campestris pv | Xanthomonas | Cotton angular leaf spot |
| malvacearum (E F Smith) | | |
| Dowson | | |
| Micrococcus flavus | Micrococcus of | Conditional pathogenic |
| | Micrococcaceae | bacteria (common indicator |
| | | bacteria) |
| Paenibacillus larvae | Paenibacillus of | American foulbrood |
| | Firmicutes | |

### (1) Activation of Pathogenic Strains

Activation of fungi: Each pathogenic fungal strain stored at 4° C. was picked by a sterilized inoculation loop, inoculated into a fresh PDA medium plate, and cultivated in a 28° C. incubator for 4 d to 5 d for later use.

Activation of bacteria: Each bacterial strain was taken, inoculated by a sterilized inoculation loop into a fresh LB solid medium, and cultivated in a 37° C. incubator for 24 h to 48 h until single colonies grew for later use. The single colonies were picked, inoculated into 3 mL of a liquid LB medium, and cultivated under shaking at 37° C. and 200 r/min for 12 h.

### (2) Determination of Inhibitory Effects of the Strains for Pathogenic Fungi

A fungal block with a diameter of 6 mm was collected by a sterilized puncher from an activated pathogenic fungal strain, and an antagonistic strain was inoculated around the fungal block at equal distances (about 3 cm away from the fungal block). A culture without any antagonistic strain was set as a control (CK). 3 replicates were set for each treatment. After 5 d to 7 d of cultivation at 28° C., an inhibition zone width (a distance between the antagonistic strain and an edge of the pathogenic fungal strain) was measured.

### (3) Determination of Inhibitory Effects of the Strains for Pathogenic Bacteria

A pathogenic bacterial strain was inoculated and cultivated until bacteria grew to a logarithmic phase to obtain a pathogenic bacterial solution. 100 μL of the pathogenic bacterial solution was taken and coated on an LB solid medium, the LB solid medium was evenly punched (each hole had a diameter of 6 mm), and then 50 μL of a filtered antagonistic bacterial solution was added to each hole. 3 replicates were set for each dish. After bacteria were cultivated at 37° C. for 1 d to 2 d, an inhibition zone diameter was measured.

### (4) Results and Analysis

Determination results of inhibitory activities of the strains QMHF-G5 and MS-E23 for plant pathogenic fungi are shown in Table 5.

TABLE 5
Determination results of inhibitory activities of the strains for plant (or bee) pathogenic fungi

| | Inhibition zone width of | Inhibition zone width |
|---|---|---|
| | the strain QMHF-G5 | of the strain MS-E23 |
| Pathogenic fungus | (mm) | (mm) |
| Botrytis cinerea (soybean Botrytis blight) | 13.93 ± 0.88 | 9.03 ± 1.31 |
| Fusarium graminearum (corn mildew and rot | 14.12 ± 1.65 | 7.43 ± 0.44 |
| diseases) | | |
| Colletotrichum orbiculare (Berk.&Mont.) Arx | 9.95 ± 0.32 | 7.43 ± 0.22 |
| (cucumber anthracnose) | | |
| Phomopsis asparagi (asparagus stem rot) | 9.68 ± 0.06 | 1.45 ± 0.34 |
| Rhizoctonia cerealis Vander Hoeven (wheat | 12.33 ± 0.29 | 5.47 ± 0.15 |
| sharp eyespot) | | |
| Cochliobolus sativus (Ito & Kurib.) Drechsler | 14.27 ± 0.54 | 8.70 ± 0.62 |
| (wheat root rot) | | |
| Valsa mali Miyabe et Yamada (apple valsa | 12.97 ± 0.34 | 8.06 ± 0.45 |
| canker) | | |
| Exserohilum turcicum (northern corn leaf blight) | 9.84 ± 1.08 | 6.84 ± 0.35 |
| Gibberella zeae (Schw.) Petch. (Fusarium head | 8.46 ± 0.36 | 5.32 ± 0.44 |
| blight) | | |
| Macrohoma kawatsukai Hara (apple ring rot) | 10.61 ± 1.01 | 6.70 ± 0.87 |
| Fusarium redolens (alfalfa root rot) | 10.07 ± 1.09 | 4.49 ± 0.45 |
| Fusarium solani (alfalfa root rot) | 11.29 ± 0.40 | 5.83 ± 0.14 |
| Fusarium Oxysporum (soybean root rot) | 8.66 ± 0.43 | 6.89 ± 0.09 |
| Magnaporthe grisea Barr (rice blast) | 11.24 ± 0.70 | 5.26 ± 0.29 |
| Alternaria panax (ginseng black spot) | 7.05 ± 0.60 | 5.09 ± 0.64 |
| Ustilaginoidea virens (rice false smut) | 7.50 ± 0.12 | 4.49 ± 0.45 |
| Aspergillus tubingensis (bee aspergillosis) | 16.74 ± 1.80 | 4.52 ± 0.24 |
| Ascosphaera apis (bee chalkbrood) | 12.32 ± 0.65 | 5.38 ± 0.37 |
| Fusarium pseudocircinatum (sunflower | 7.49 ± 1.41 | 3.28 ± 0.85 |
| malformation) | | |
| Mucor irregularis (strawberry root rot) | 7.85 ± 1.09 | 4.93 ± 0.83 |

It can be seen from Table 5 that the strain QMHF-G5 has a significant inhibitory effect for 20 pathogenic fungus strains, where the strain QMHF-G5 has an optimal inhibitory effect for Aspergillus tubingensis with an inhibition zone width of 16.74 mm, the strain QMHF-G5 has an inhibition zone width of 14.27 mm for Cochliobolus sativus (Ito & Kurib.) Drechsler, the strain QMHF-G5 also exhibits a relatively-high inhibitory activity for Fusarium graminearum when co-cultivated with Fusarium graminearum and has an inhibition zone width of 14.12 mm, the strain QMHF-G5 has an inhibition zone width of 13.93 mm for Botrytis cinerea and an inhibition zone width of 12.97 mm for Valsa mali Miyabe et Yamada, the strain QMHF-G5 has an inhibition zone width of greater than 10 mm for more than half of the target fungi in this experiment, and the strain QMHF-G5 also exhibits a significant inhibitory ability against other test fungi.

The strain MS-E23 exhibits a slightly-weak inhibitory effect for the 20 pathogenic fungus strains, where the strain MS-E23 has a most significant inhibitory effect on Botrytis cinerea with an inhibition zone width of 9.03 mm, the strain MS-E23 has an inhibition zone width of 8.70 mm for Cochliobolus sativus (Ito & Kurib.) Drechsler and an inhibition zone width of 8.06 mm for Valsa mali Miyabe et Yamada, and the strain MS-E23 exhibits a relatively-weak inhibitory ability against the remaining 17 strains.

Determination results of inhibitory activities of the strains QMHF-G5 and MS-E23 for pathogenic bacteria are shown in Table 6.

TABLE 6
Determination results of inhibitory activities of the strains for pathogenic bacteria

| | Inhibition zone | Inhibition zone |
|---|---|---|
| | diameter of the | diameter of the |
| | strain QMHF-G5 | strain MS-E23 |
| Pathogenic bacteria | (mm) | (mm) |
| Xanthomonas oryzae pv. oryzae (rice bacterial | 29.5 ± 0.14 | — |
| leaf blight) | | |
| Pseudomonas solanacearum Smith (peanut | 14.09 ± 0.86 | — |
| bacterial wilt) | | |
| Xanthomonas campestris pv malvacearum (E | 18.95 ± 0.28 | 9.11 ± 1.48 |
| F Smith) Dowson (cotton angular leaf spot) | | |
| Paenibacillus larvae (American foulbrood) | 34.62 ± 0.12 | 18.41 ± 1.07 |
| Micrococcus flavus conditional pathogenic | 38.51 ± 0.15 | 19.48 ± 0.16 |
| bacteria (common indicator bacteria) | | |

It can be seen from Table 6 that the strain QMHF-G5 exhibits different inhibitory activities against different pathogenic bacteria, where the strain QMHF-G5 has an inhibition zone diameter of 38.51 mm for Micrococcus flavus and an inhibition zone diameter of 34.62 mm for Paenibacillus larvae, and the strain QMHF-G5 also exhibits strong inhibitory activities for other strains detected.

The strain MS-E23 has no inhibitory effect on both Xanthomonas oryzae pv. oryzae and Pseudomonas solanacearum Smith, and has an inhibition zone diameter of 19.48 mm for Micrococcus flavus, an inhibition zone diameter of 18.41 mm for Paenibacillus larvae, and an inhibition zone diameter of 9.11 mm for Xanthomonas campestris pv malvacearum (EF Smith) Dowson.

The strain QMHF-G5 not only has a significant inhibitory effect on a wide range of fungal species, but also exhibits an effective inhibitory activity for all pathogenic bacteria detected. In recent years, the search for effective biological control strains in the field of biological control has become a key to the sustainable agricultural development. The results of the present disclosure indicate that the B. velezensis QMHF-G5 has a huge application potential in the control and prevention of various diseases caused by fungi and bacteria. With the in-depth research on the action mechanism, safety, and application effect of the B. velezensis QMHF-G5, the B. velezensis QMHF-G5 is expected to become an important microorganism against a variety of pathogens and make important contributions to the human health and environmental protection.

The above are merely preferred specific implementations of the present disclosure, but the protection scope of the present disclosure is not limited thereto. Any modification or replacement easily conceived by those skilled in the art within the technical scope of the present disclosure should fall within the protection scope of the present disclosure.

What is claimed is:

1. Bacillus velezensis (B. velezensis) QMHF-G5 with an accession number of CGMCC No. 27713.

2. The B. velezensis QMHF-G5 according to claim 1, wherein the B. velezensis QMHF-G5 comprises 16S rDNA shown in SEQ ID NO: 1.

3. The B. velezensis QMHF-G5 according to claim 1, wherein the B. velezensis QMHF-G5 comprises a QMHF-G5gyrB gene sequence shown in SEQ ID NO: 2.

4. The B. velezensis QMHF-G5 according to claim 1, wherein the B. velezensis QMHF-G5 is isolated from an intestinal tract of Vespula flaviceps.

5. The B. velezensis QMHF-G5 according to claim 1, wherein the B. velezensis QMHF-G5 has an inhibition zone width of more than or equal to 16.74 mm for Aspergillus tubingensis, an inhibition zone width of more than or equal to 14.27 mm for Cochliobolus sativus (Ito & Kurib.) Drechsler, an inhibition zone width of more than or equal to 14.12 mm for Fusarium graminearum, an inhibition zone width of more than or equal to 13.93 mm for Botrytis cinerea, and an inhibition zone width of more than or equal to 12.97 mm for Valsa mali Miyabe et Yamada.

6. The B. velezensis QMHF-G5 according to claim 1, wherein the B. velezensis QMHF-G5 has an inhibition zone diameter of more than or equal to 38.51 mm for Micrococcus flavus and an inhibition zone diameter of more than or equal to 34.62 mm for Paenibacillus larvae.

7. A biological agent or inhibition agent for inhibiting fungi and bacteria, comprising the B. velezensis QMHF-G5 with an accession number of CGMCC No. 27713.

8. The biological agent or inhibition agent according to claim 7, wherein the fungi comprise Botrytis cinerea, Fusarium graminearum, Colletotrichum orbiculare (Berk. & Mont.) Arx, Phomopsis asparagi, Rhizoctonia cerealis Vander Hoeven, Cochliobolus sativus (Ito & Kurib.) Drechsler, Valsa mali Miyabe et Yamada, Exserohilum turcicum, Gibberella zeae (Schw.) Petch., Macrohoma kawatsukai Hara, Fusarium redolens, Fusarium solani, Fusarium Oxysporum, Magnaporthe grisea Barr, Alternaria panax, Ustilaginoidea virens, Aspergillus tubingensis, Ascosphaera apis, Fusarium pseudocircinatum, and Mucor irregularis.

9. The biological agent or inhibition agent according to claim 7, wherein the bacteria comprise Xanthomonas oryzae pv. oryzae, Pseudomonas solanacearum Smith, Xanthomonas campestris pv malvacearum (E F Smith) Dowson, Paenibacillus larvae, and Micrococcus flavus.

10. A use of the B. velezensis QMHF-G5 with an accession number of CGMCC No. 27713 in preparation of a drug for controlling animal and plant diseases.
